(19) United States
(12) Patent Application Publication
(10) Pub. No.: US 2024/0415464 A1
(43) Pub. Date: Dec. 19, 2024

(54) MENTAL DISORDER DETERMINATION DEVICE, TERMINAL DEVICE, CLASSIFIER, MENTAL DISORDER DETERMINATION ASSISTANCE METHOD, AND RECORDING MEDIUM

(71) Applicants: KEIO UNIVERSITY, Tokyo (JP); TEIJIN PHARMA LIMITED, Tokyo (JP)

(72) Inventors: Yoshihiro NODA, Tokyo (JP); Kento Sakaue, Osaka-shi (JP)

(73) Assignees: KEIO UNIVERSITY, Tokyo (JP); TEIJIN PHARMA LIMITED, Tokyo (JP)

(21) Appl. No.: 18/498,680

(22) Filed: Oct. 31, 2023

(30) Foreign Application Priority Data
Jun. 15, 2023 (JP) 2023-098482

(51) Int. Cl. A61B 5/00; A61B 5/256; A61B 5/273; A61B 5/291; A61B 5/374; A61B 5/384; A61B 5/386

(52) U.S. Cl. CPC A61B 5/7267; A61B 5/256; A61B 5/273; A61B 5/291; A61B 5/374; A61B 5/384; A61B 5/386; A61B 5/742; A61B 2560/0468

(57) ABSTRACT

A feature amount extractor extracts a feature amount representing the feature of a brain wave from the brain wave of a subject, the brain wave being measured when a magnetic stimulation is applied to the brain of the subject. A mental disorder determiner determines whether or not the subject has a predetermined mental disorder on the basis of a feature amount variation that is a differential value between a feature amount extracted from a brain wave measured immediately before the magnetic stimulation and a feature amount extracted from a brain wave measured immediately after the magnetic stimulation.

### CROSS-REFERENCE TO RELATED APPLICATIONS

This application claims the benefit of Japanese Patent Application No. 2023-098482, filed on Jun. 15, 2023, the entire disclosure of which is incorporated by reference herein.

### FIELD OF THE INVENTION

The present disclosure relates to a mental disorder determination device, a terminal device, a classifier, a mental disorder determination assistance method, and a non-transitory recording medium.

### BACKGROUND OF THE INVENTION

Currently, the presence or absence of mental disorders is often determined using indicators based on clinical symptoms and the like appearing in subjects as a result of medical examination of the subjects by interview. However, such indicators are not able to be considered to be highly objective indicators, and preclude enhancement of the accuracy of determination of mental disorder diagnosis. Therefore, techniques to determine the presence or absence of mental disorders using highly objective indicators have been researched.

An indicator based on a brain wave in quiet wakefulness or after transcranial magnetic stimulation is known as such an indicator. For example, International Publication No. WO 2018/066715 describes a technique to determine the presence or absence of a mental disorder on the basis of a brain wave in a plurality of sites in the brain after transcranial magnetic stimulation.

### SUMMARY OF THE INVENTION

However, the enhancement of determination accuracy is difficult in a method in which the presence or absence of a mental disorder is determined based on only a single brain wave component after transcranial magnetic stimulation by a single electrode, as described in International Publication No. WO 2018/066715. Likewise, the enhancement of determination accuracy is also difficult in a method in which the presence or absence of a mental disorder is determined based on only a specific brain wave component in quiet wakefulness by a single electrode. Therefore, a technique to accurately determine the presence or absence of a mental disorder using an objective indicator is desired.

The present disclosure was made in view of the problems described above and is directed at providing a mental disorder determination device and the like by which the presence or absence of a mental disorder is accurately determined using an objective indicator.

In order to achieve the objective described above, a mental disorder determination device according to aspect 1 of the present disclosure includes processing circuitry to
extract a feature amount that represents a feature of a brain wave of a subject from the brain wave measured when a magnetic stimulation is applied to a brain of the subject, and
determine whether or not the subject has a predetermined mental disorder based on a feature amount variation that is a differential value between a feature amount extracted from a brain wave measured immediately before the magnetic stimulation and a feature amount extracted from a brain wave measured immediately after the magnetic stimulation.

The magnetic stimulation may be consecutively applied to the brain of the subject, and
the feature amount variation may be a differential value between a feature amount extracted from a brain wave measured immediately before a second or later magnetic stimulation and a feature amount extracted from a brain wave measured immediately after the second or later magnetic stimulation.

The brain waves may be brain waves at a plurality of measurement spots of the subject, and
the feature amount may include at least one of a frequency power value of at least one of α, β, γ, θ, and δ waves of each brain wave at the measurement spots, a phase synchronization value of at least one of α, β, γ, θ, and δ waves of each brain wave between the measurement spots, or a phase-amplitude coupling value in at least one combination of a plurality of combinations of a phase of an α, θ, or δ wave and an amplitude of a β or γ wave of a brain wave at a specific measurement spot.

The brain waves may be brain waves at a plurality of measurement spots of the subject, and
the feature amount may include a frequency power value of at least one of α, β, γ, θ, and δ waves of each brain wave at the measurement spots, a phase synchronization value of at least one of α, β, γ, θ, and δ waves of each brain wave between the measurement spots, and a phase-amplitude coupling value in at least one combination of a plurality of combinations of a phase of an α, θ, or δ wave and an amplitude of a β or γ wave of a brain wave at a specific measurement spot.

The mental disorder may be major depressive disorder or treatment-resistant depression.

The brain waves may be brain waves at a plurality of measurement spots on a forehead of the subject, the brain waves being measured through a plurality of electrodes placed on the forehead of the subject.

The processing circuitry may determine whether or not the subject has the mental disorder using a learned model into which the feature amount variation is input to output a result of determining whether or not the subject has the mental disorder.

The processing circuitry may determine whether or not the subject has the mental disorder based on the feature amount variation and on a feature amount extracted from a brain wave measured in quiet wakefulness.

The processing circuitry may determine whether or not the subject has the mental disorder based on the feature amount variation and on a feature amount extracted from a brain wave measured immediately before the magnetic stimulation.

The processing circuitry may determine whether or not the subject has the mental disorder based on the feature amount variation and on a feature amount extracted from a brain wave measured immediately after the magnetic stimulation.

The processing circuitry may determine whether or not the subject has the mental disorder based on the feature amount variation, on a feature amount extracted from a brain wave measured in quiet wakefulness, and on a feature amount extracted from a brain wave measured immediately before the magnetic stimulation.

The processing circuitry may determine whether or not the subject has the mental disorder based on the feature amount variation, on a feature amount extracted from a brain wave measured in quiet wakefulness, and on a feature amount extracted from a brain wave measured immediately after the magnetic stimulation.

The processing circuitry may determine whether or not the subject has the mental disorder based on the feature amount variation, on a feature amount extracted from a brain wave measured immediately before the magnetic stimulation, and on a feature amount extracted from a brain wave measured immediately after the magnetic stimulation.

The processing circuitry may determine whether or not the subject has the mental disorder based on the feature amount variation, on a feature amount extracted from a brain wave measured in quiet wakefulness, on a feature amount extracted from a brain wave measured immediately before the magnetic stimulation, and on a feature amount extracted from a brain wave measured immediately after the magnetic stimulation.

In order to achieve the objective described above, a terminal device according to aspect 2 of the present disclosure includes processing circuitry to
acquire brain wave data indicating a brain wave of a subject, the brain wave being measured when a magnetic stimulation is applied to the brain of the subject,
transmit the acquired brain wave data to a server, and
cause a display to display a result of determining whether or not the subject has a predetermined mental disorder, the result being determined, by the server, based on a feature amount variation that is a differential value between a feature amount extracted from a brain wave measured immediately before the magnetic stimulation and a feature amount extracted from a brain wave measured immediately after the magnetic stimulation.

In order to achieve the objective described above, a classifier according to aspect 3 of the present disclosure
receives a feature amount variation that is a differential value between a feature amount extracted from a brain wave measured immediately before a magnetic stimulation to a brain of a subject and a feature amount extracted from a brain wave measured immediately after the magnetic stimulation, and
outputs a result of determining whether or not the subject has a predetermined mental disorder.

In order to achieve the objective described above, a mental disorder determination assistance method according to aspect 4 of the present disclosure is a mental disorder determination assistance method for assisting determination of presence or absence of a mental disorder, the method including:
extracting a feature amount representing a feature of a brain wave of a subject from the brain wave measured when a magnetic stimulation is applied to a brain of the subject, and
determining whether or not the subject has a predetermined mental disorder based on a feature amount variation that is a differential value between a feature amount extracted from a brain wave measured immediately before the magnetic stimulation and a feature amount extracted from a brain wave measured immediately after the magnetic stimulation.

In order to achieve the objective described above, in a non-transitory computer readable recording medium according to aspect 5 of the present disclosure stores a program, the program causing a computer to function as
a feature amount extractor that extracts a feature amount representing a feature of a brain wave of a subject from the brain wave measured when a magnetic stimulation is applied to a brain of the subject, and
a mental disorder determiner that determines whether or not the subject has a predetermined mental disorder based on a feature amount variation that is a differential value between a feature amount extracted from a brain wave measured immediately before the magnetic stimulation and a feature amount extracted from a brain wave measured immediately after the magnetic stimulation.

In accordance with the present disclosure, the presence or absence of a mental disorder can be accurately determined using an objective indicator.

### BRIEF DESCRIPTION OF DRAWINGS

A more complete understanding of this application can be obtained when the following detailed description is considered in conjunction with the following drawings, in which:

FIG. 1 is a configuration view of a mental disorder determination system according to Embodiment 1;

FIG. 2 is a configuration view of a mental disorder determination device according to Embodiment 1;

FIG. 3 is a view illustrating brain waves before and after a magnetic stimulation;

FIG. 4 is an explanatory diagram of consecutive magnetic stimulations;

FIG. 5 is a functional configuration view of the mental disorder determination device according to Embodiment 1;

FIG. 6 is a flow chart illustrating a mental disorder determination process executed by a mental disorder determination device according to an embodiment;

FIG. 7 is a flow chart illustrating a feature amount extraction process illustrated in FIG. 6;

FIG. 8 is a view illustrating the results of evaluation of learned models;

FIG. 9 is a configuration view of a mental disorder determination system according to Embodiment 2;

FIG. 10 is a configuration view of a mental disorder determination device according to Embodiment 2;

FIG. 11 is a configuration view of a terminal device according to Embodiment 2; and

FIG. 12 is a functional configuration view of the mental disorder determination device and the terminal device according to Embodiment 2.

### DETAILED DESCRIPTION OF THE INVENTION

Embodiments of the present disclosure are described below with reference to the drawings. In the drawings, the same or corresponding portions are denoted by the same reference characters.

### Embodiment 1

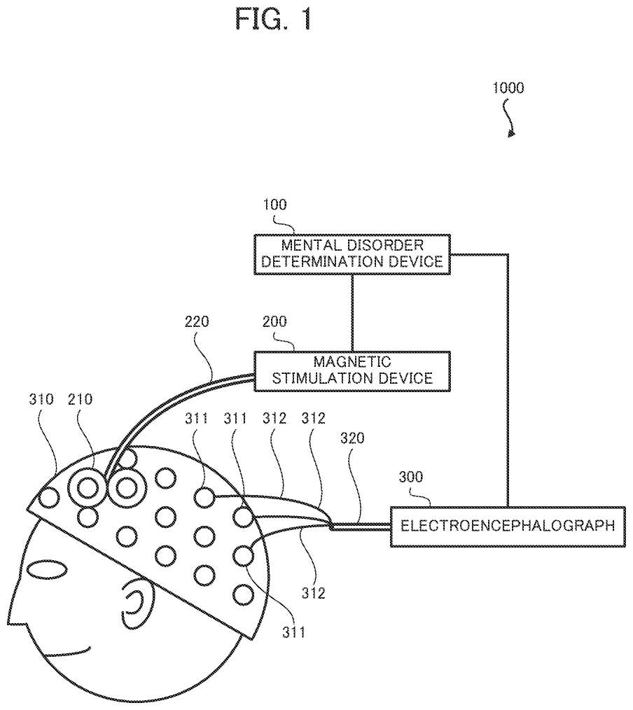

First, the configuration of a mental disorder determination system 1000 according to the present embodiment is described with reference to FIG. 1. The mental disorder determination system 1000 is a system that determines whether or not a subject has a mental disorder on the basis of the brain wave of the subject. Examples of the mental disorder include treatment-resistant depression, major depressive disorder, bipolar disorder, schizophrenia, dementia, Alzheimer's disease, anxiety disorder, and epilepsy. The treatment-resistant depression is a depression for which drugs are inhibited from being effective, and against which even use of sufficient doses of one or more kinds of antidepressant drugs for a sufficient period provides no antidepressant effect. The major depressive disorder is a so-called common depression that is a mental disorder in which a psychiatric symptom such as depressive mood or hypobulia, and a physical symptom such as anorexia, insomnia, or fatigue continue for two or more weeks.

The bipolar disorder is a brain disease in which a manic state and a depressive state are repeated. The dementia is a disease in which a cognitive function including memory is deteriorated due to various causes, whereby daily life, social life, and the like are impaired. The anxiety disorder is a disease that produces a feeling of such strong anxiety, fear, or the like that a problem in daily life occurs. Epilepsy is a disease in which temporarily excessive excitement of the brain causes epileptic seizure to appear in a brain wave, whereby consciousness is lost, or convulsions occur. The mental disorder determination system 1000 includes a mental disorder determination device 100, a magnetic stimulation device 200, and an electroencephalograph 300.

The mental disorder determination device 100 is a device that determines whether or not a subject has a mental disorder on the basis of the brain wave of the subject. It is considered that between a person who has a mental disorder caused by some kind of disorder in the brain and a person who does not have the mental disorder, there are differences in a quiet wakefulness brain wave that is a brain wave in quiet wakefulness, a pre-stimulus brain wave that is a brain wave immediately before magnetic stimulation, a post-stimulus brain wave that is a brain wave immediately after magnetic stimulation, the ways of appearance of a brain wave change before and after magnetic stimulation, an intermediate phenotype, and the like. In the present embodiment, the mental disorder determination device 100 determines whether or not a subject has a predetermined mental disorder on the basis of the pre-stimulus brain wave and the post-stimulus brain wave. The predetermined mental disorder means at least one previously specified mental disorder among mental disorders.

The state of quiet wakefulness refers to a state in which a subject relaxes and is waking for a predetermined time or longer. The magnetic stimulation is a magnetic stimulation that is applied to the brain of the subject. In the present embodiment, magnetic stimulations are repeatedly applied to the brain of the subject, and brain waves before and after the second or later magnetic stimulation is applied are used. In other words, in the present embodiment, the pre-stimulus brain wave is the brain wave of the subject immediately before the second or later magnetic stimulation is applied, and the post-stimulus brain wave is the brain wave of the subject immediately after the second or later magnetic stimulation is applied. The intensity of a magnetic stimulation, the time of a magnetic stimulation, and the like can be appropriately set in reference to common knowledge in the present field.

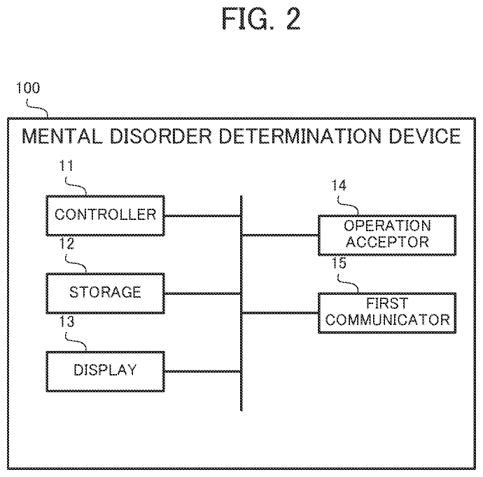

The mental disorder determination device 100 controls the magnetic stimulation device 200 to apply a magnetic stimulation to the brain of a subject. The mental disorder determination device 100 acquires brain wave data indicating the brain wave of the subject from the electroencephalograph 300. The mental disorder determination device 100 can communicate with the magnetic stimulation device 200 and the electroencephalograph 300. As illustrated in FIG. 2, the mental disorder determination device 100 includes a controller 11, a storage 12, a display 13, an operation acceptor 14, and a first communicator 15.

The controller 11 includes a central processing unit (CPU), a read only memory (ROM), a random access memory (RAM), a real time clock (RTC), and the like. The CPU is also referred to as a central processing unit, a central arithmetic device, a processor, a microprocessor, a microcomputer, a digital signal processor (DSP), or the like, and functions as a central arithmetic processing device that executes a process and arithmetic according to control of the mental disorder determination device 100. In the controller 11, the CPU reads a program and data stored in the ROM, and integrally controls the mental disorder determination device 100 using the RAM as a work area. For example, the RTC is an integrated circuit including a time measurement function. The CPU can specify current date and time on the basis of time information read from the RTC.

The storage 12 includes a nonvolatile semiconductor memory such as a flash memory, an erasable programmable ROM (EPROM), or an electrically erasable programmable ROM (EEPROM), and plays a role as a so-called auxiliary storage device. The storage 12 stores a program and data used for allowing the controller 11 to execute various processes. Moreover, the storage 12 stores data generated and acquired by the execution of the various processes by the controller 11.

The display 13 displays various images under control by the controller 11. For example, the display 13 displays a screen for accepting various operations from a user. The display 13 includes a touch screen, a liquid crystal display, and/or the like. The operation acceptor 14 accepts the various operations from the user, and supplies information indicating the contents of the accepted operations to the controller 11. The operation acceptor 14 includes a touch screen, a button, and a lever, and/or the like.

The first communicator 15 communicates with various devices under control by the controller 11. In the present embodiment, the first communicator 15 communicates with the magnetic stimulation device 200 and the electroencephalograph 300 under control by the controller 11. The first communicator 15 communicates with various devices in conformity with a well-known wire communication standard or a well-known wireless communication standard. Examples of the well-known wire communication standard include Universal Serial Bus (USB, registered trademark) and Thunderbolt (registered trademark). Examples of the well-known wireless communication standard include Wi-Fi (registered trademark), Bluetooth (registered trademark), and Zigbee (registered trademark). The first communicator 15 includes a communication interface conforming to various communication standards.

The magnetic stimulation device 200 is a device that applies transcranial magnetic stimulation (TMS), that is, transcranial magnetic stimulation to a subject. The magnetic stimulation device 200 is operated, for example, under control by the mental disorder determination device 100. Accordingly, the magnetic stimulation device 200 includes a communication interface (not illustrated) for communicating with the mental disorder determination device 100. The magnetic stimulation device 200 is connected to a coil 210 through a cable 220. The magnetic stimulation device 200 sharply changes a current allowed to flow into the coil 210, and sharply changes a magnetic field around the coil 210. The magnetic stimulation device 200 induces a current in the brain by the sharp change of the magnetic field, to excite neurons in the brain.

The coil 210 sharply changes a magnetic field around the coil 210 depending on the sharp change of the current flowing into the coil 210. The magnetic field passes without being obstructed by the scalp, the cranial bone, and the like, and induces, in the brain, a current that cancels the change of the magnetic field. Like electric stimulation to the surface of the cortex, the current induced in the brain activates nerve cells in the vicinity thereof. The shape of the coil 210 is, for example, a figure-of-eight flat shape. The coil 210 is attached to the head of a subject so that the plane of the coil 210 and a tangential direction with respect to the cranial bone are orthogonal to each other. The coil 210 is attached to a portion corresponding to, for example, the prefrontal area (for example, the left dorsolateral prefrontal area) of the subject.

The electroencephalograph 300 is a device that measures the electroencephalogram (EEG) of a subject, that is, the brain wave of the subject. The electroencephalograph 300 is operated, for example, under control by the mental disorder determination device 100. Accordingly, the electroencephalograph 300 includes a communication interface (not illustrated) for communicating with the mental disorder determination device 100. The electroencephalograph 300 is connected to an electroencephalography cap 310 through a cable 320.

The electroencephalography cap 310 is a cap that is attached to the head of a subject in order to measure the brain wave of the subject. The electroencephalography cap 310 includes a plurality of electrodes 311 for detecting the brain wave of the subject. The plurality of respective electrodes 311 is disposed at positions on the electroencephalography cap 310, the positions corresponding to a plurality of measurement spots on the head of the subject. In the present embodiment, the plurality of electrodes 311 is placed on the whole electroencephalography cap 310, and a brain wave is measured on the whole or part of the head of the subject.

The respective electrodes 311 are connected to the electroencephalograph 300 through electric wires 312 packed in the cable 320. The electroencephalograph 300 measures a brain wave at each measurement spot by measuring the potential of each electrode 311. In FIG. 1, the reference numeral and electric wires 312 are illustrated only for some electrodes 311. The numbers of the spots at which the brain wave is measured and the electrodes by which the brain wave is measured are optional. In the present embodiment, the total number of the spots at which the brain wave is measured is 62 on the whole head, with regard to the number of the electrodes by which the brain wave is measured. In the present embodiment, the term “channel” is appropriately used in the case of specifying the electrodes 311, the measurement spots, and the like.

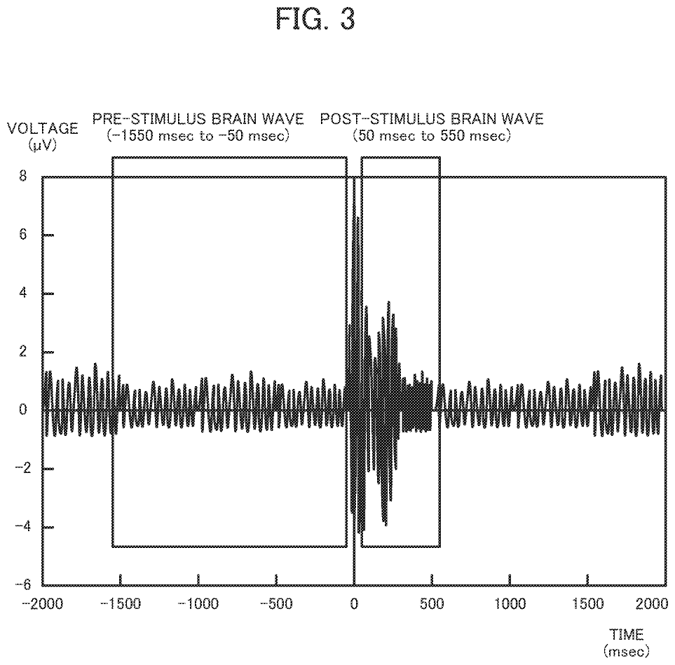

A brain wave measured by the electroencephalograph 300 is described with reference to FIG. 3. A brain wave is an electrical signal generated by the brain of a human. The brain wave can be measured, for example, by detecting the potentials of the electrodes 311 placed on the scalp. In the present embodiment, a time-series voltage signal indicating a brain wave is appropriately referred to as a brain wave or a brain wave signal, data indicating a time-series voltage signal indicating a brain wave is appropriately referred to as brain wave data.

As illustrated in FIG. 3, the brain wave is expressed as a signal indicating a time variation in voltage. FIG. 3 illustrates brain waves before and after a magnetic stimulation is applied to the brain of a subject in a case in which timing at which the magnetic stimulation is applied to the brain of the subject is set at 0 msec. The amplitudes of the brain waves are magnitudes of around several microvolts to several tens of microvolts. In the present embodiment, the electroencephalograph 300 measures the voltage of a brain wave at a sampling frequency of, for example, 3 kHz, and the brain wave data is data indicating a time-series voltage measured at a sampling frequency of, for example, 3 kHz.

FIG. 3 illustrates a pre-stimulus brain wave that is a brain wave immediately before the magnetic stimulation, and a post-stimulus brain wave that is a brain wave immediately after the magnetic stimulation. In the present embodiment, the pre-stimulus brain wave is a brain wave from 1550 msec before the magnetic stimulation to 50 msec before the magnetic stimulation. The post-stimulus brain wave is a brain wave from 50 msec after the magnetic stimulation to 550 msec after the magnetic stimulation. From 50 msec before a magnetic stimulation to 50 msec after the magnetic stimulation, large noise is highly likely to be superposed on a measured brain wave. Thus, in the present embodiment, a brain wave from 50 msec before a magnetic stimulation to 50 msec after the magnetic stimulation is not included in the pre-stimulus brain wave and the post-stimulus brain wave in principle. FIG. 3 does not illustrate a quiet wakefulness brain wave that is a brain wave in quiet wakefulness. The quiet wakefulness brain wave is, for example, a several-second brain wave measured in quiet wakefulness.

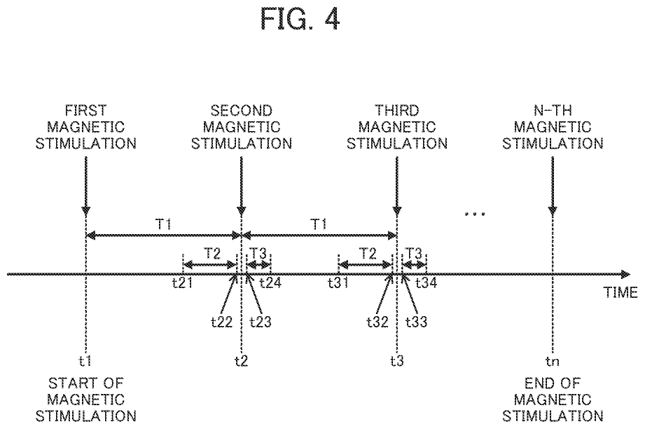

In the present embodiment, magnetic stimulations are repeatedly executed from t1 to tn, as illustrated in FIG. 4. Specifically, the first magnetic stimulation is executed at t1, the second magnetic stimulation is executed at t2 after a lapse of T1 from t1, and the third magnetic stimulation is executed at t3 after a lapse of T1 from t2. Afterward, magnetic stimulations are repeatedly executed at intervals of T1, and the n-th magnetic stimulation is executed at tn. T1 is, for example, several seconds.

In the present embodiment, the pre-stimulus brain wave is a brain wave immediately before a magnetic stimulation in a case in which magnetic stimulations are repeated, and the post-stimulus brain wave is a brain wave immediately after the magnetic stimulation in a case in which the magnetic stimulations are repeated. In other words, in the present embodiment, the pre-stimulus brain wave is a brain wave immediately before the second or later magnetic stimulation, and the post-stimulus brain wave is a brain wave immediately after the second or later magnetic stimulation. Accordingly, the pre-stimulus brain wave is definitely distinguished from a quiet wakefulness brain wave in the present embodiment.

The pre-stimulus brain wave may be a brain wave immediately before the second magnetic stimulation, and the post-stimulus brain wave may be a brain wave immediately after the second magnetic stimulation. In this case, the pre-stimulus brain wave is a brain wave from t21 to t22, and the post-stimulus brain wave is a brain wave from t23 to t24. For example, t21 is time 1550 msec before t2, and t22 is, for example, time 50 msec before t2. For example, t23 is time 50 msec after t2, and t24 is, for example, time 550 msec after t2.

Alternatively, the pre-stimulus brain wave may be a brain wave immediately before the third magnetic stimulation, and the post-stimulus brain wave may be a brain wave immediately after the third magnetic stimulation. In this case, the pre-stimulus brain wave is a brain wave from t31 to t32, and the post-stimulus brain wave is a brain wave from t33 to t34. For example, t31 is time 1550 msec before t3, and t32 is, for example, time 50 msec before t3. For example, t33 is time 50 msec after t3, and t34 is, for example, time 550 msec after t3. T2 that is time from t21 to t22, and, for example, time from t31 to t32 is 1500 msec, and T3 that is time from t23 to t24, and, for example, time from t33 to t34 is 500 msec.

The pre-stimulus brain wave may be a brain wave formed by averaging brain waves immediately before the plurality of second or later magnetic stimulations, and the post-stimulus brain wave may be a brain wave formed by averaging brain waves immediately after the plurality of second or later magnetic stimulations. Averaging of brain waves means that the time axes of the respective brain waves are adjusted so that the timings of magnetic stimulations occur at the same time, and the average value of voltages is determined every elapsed time before the timing of each magnetic stimulation or every elapsed time after the timing of each magnetic stimulation.

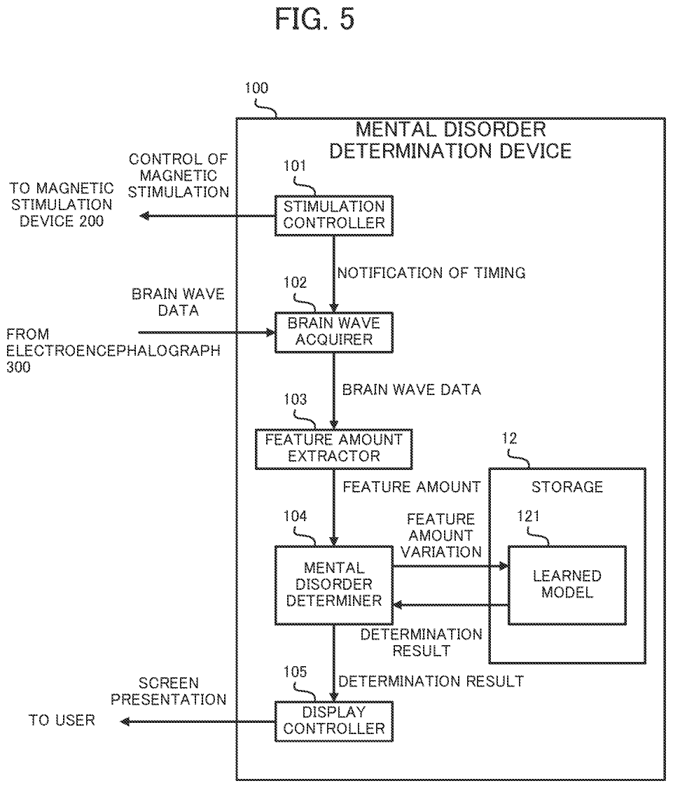

Referring now to FIG. 5, the functions of the mental disorder determination device 100 are described. The mental disorder determination device 100 functionally includes a stimulation controller 101, a brain wave acquirer 102, a feature amount extractor 103, a mental disorder determiner 104, and a display controller 105. Each function thereof is implemented by software, firmware, or a combination of software and firmware. The software and the firmware are described as programs, and stored in ROM or the storage 12. The CPU executes the programs stored in the ROM or the storage 12, thereby implementing each function thereof.

The stimulation controller 101 controls a magnetic stimulation to the brain of a subject. For example, the stimulation controller 101 controls the magnetic stimulation device 200 to apply magnetic stimulations to the brain of the subject at predetermined time intervals. The stimulation controller 101 and the brain wave acquirer 102 are preferably synchronized as appropriate. For example, the stimulation controller 101 may notify the brain wave acquirer 102 of the timing of applying a magnetic stimulation to the brain of the subject.

The brain wave acquirer 102 acquires the brain wave of the subject. For example, the brain wave acquirer 102 controls the electroencephalograph 300 to measure the brain wave of the subject, and acquires brain wave data indicating the measured brain wave from the electroencephalograph 300. For example, the brain wave acquirer 102 acquires a pre-stimulus brain wave and a post-stimulus brain wave at all the measurement spots on the head of the subject. The brain wave acquirer 102 may extract the pre-stimulus brain wave and the post-stimulus brain wave from the brain wave indicated by the brain wave data acquired from the electroencephalograph 300. Alternatively, the brain wave acquirer 102 may acquire brain wave data indicating the pre-stimulus brain wave and brain wave data indicating the post-stimulus brain wave from the electroencephalograph 300.

The feature amount extractor 103 extracts a feature amount representing the feature of a brain wave from the brain wave of a subject, the brain wave being measured when a magnetic stimulation is applied to the brain of the subject. In other words, the feature amount extractor 103 extracts a feature amount representing the feature of the brain wave of the subject from the brain wave indicated by the brain wave data acquired by the brain wave acquirer 102. What is adopted as the feature amount can be adjusted as appropriate. For example, the frequency power value of at least one of the α, β, γ, θ, and δ waves of a brain wave at each measurement spot may be adopted as the feature amount.

The α wave is the pattern of a brain wave that is often observed in a quiet, closed-eye, and wakeful state, and is a component between 8 Hz and 13 Hz among brain waves. The β wave is the pattern of a brain wave associated with usual consciousness at the time of wakefulness, and is a component between 14 Hz and 30 Hz among brain waves. The γ wave is the pattern of a brain wave associated with perception, consciousness, and/or the like, and is generated by synchronization activity performed by a nerve cell population. The γ wave is a component of 30 Hz or more among brain waves. The θ wave is seen in the case of relaxation, is the pattern of a brain wave associated with a cognitive function and/or the like, and is a component between 4 Hz and 7 Hz among brain waves. The δ wave is the pattern of a brain wave associated with slow wave sleep, and is a component between 0.5 Hz and 3 Hz among brain waves. The frequency power is the power in a corresponding frequency band.

The frequency power can be determined from, for example, a power spectrum acquired by fast Fourier transform (FFT) analysis of a brain wave that is a time-series electrical signal. For example, the frequency power of the α wave is a power in a frequency band between 8 Hz and 13 Hz among powers indicated by power spectra. The frequency power value and a variation in the frequency power value are considered to differ between a person with a mental disorder and a person with no mental disorder. Thus, the frequency power value is adopted as the feature amount.

The phase synchronization value of at least one of the α, β, γ, θ, and δ waves of each brain wave between the measurement spots may also be adopted as the feature amount. The phase synchronization indicates the degree of the synchronization of the brain waves in two regions. In other words, the phase synchronization indicates such a degree that the peaks and troughs of the corresponding frequency components of the brain waves measured at two measurement spots overlap. The phase synchronization value is determined on the basis of a pair of measurement spots, and on the basis of the frequency bands of the α, β, γ, θ, and δ waves. For example, in a case in which the number of measurement spots is 62, phase synchronization values of which the number is ₆₂C₂×5=9455 are calculated.

The phase synchronization value can be calculated by, for example, Equation (1) described below using a weighted phase lag index (wPLI). The wPLI indicates the degree of the distribution of a phase angle difference between x (t) and y (t) that are two time-series real number signals toward the positive or negative portion of the imaginary axis of the complex plane. S_{xy} is the complex cross-spectral density of x (t) and y (t). In addition, imag is a function that returns the imaginary part of a complex. In addition, sgn is a signum function that returns any of −1, 0, and 1 depending on the sign of a real number. The coupling value of single wPLI in each of frequency bands (frequency bands of α, β, γ, θ, and δ waves) can be calculated by calculating the average value of the whole frequency bin of the frequency range of an object.

$$[ Math 1 ]$$

$$wPLI = \vert \frac{{\sum }_{t = 1}^{n} \vert imag ( {S}_{xy , t} ) \vert sgn ( imag ( {S}_{x y , t} ) )}{{\sum }_{t = 1}^{n} \vert ima g ( {S}_{x y , t} ) \vert} \vert \qquad ( 1 )$$

A phase synchronization value and a variation in the phase synchronization value are considered to differ between a person with a mental disorder and a person with no mental disorder. For example, a person with a mental disorder has deteriorated functional integration, and is therefore considered to have a decreased phase synchronization value. Thus, a phase synchronization value is adopted as the feature amount.

The value of the coupling of the phase of the α wave and the amplitude of the γ wave of a brain wave at a specific measurement spot may be adopted as the feature amount. The specific measurement spot is, for example, a measurement spot corresponding to a site to which a magnetic stimulation is applied by the coil 210, and, for example, a measurement spot corresponding to the prefrontal area (for example, left dorsolateral prefrontal area). Coupling of the phase of a low-frequency component and the amplitude of a high-frequency component means that the phase of the low-frequency component influences the amplitude of the high-frequency component, and that the phase of an envelope represented by the amplitude of the high-frequency component coincides with the phase of the low-frequency component. In such a case, the α wave is the low-frequency component, and the γ wave is the high-frequency component. A method in which the feature amount extractor 103 calculates the value of the coupling of the phase of the α wave and the amplitude of the γ wave is described below.

First, the feature amount extractor 103 extracts the signal of the α wave from the brain wave signal indicated by the brain wave data using a band-pass filter through which the frequency band of the α wave is passed. Moreover, the feature amount extractor 103 extracts the signal of the γ wave from the brain wave signal using a band-pass filter through which the frequency band of the γ wave is passed. The feature amount extractor 103 generates an envelope signal from the signal of the γ wave. The envelope signal is a signal indicating the magnitude of the amplitude of the γ wave at each time, and a signal indicating the distance between the upper and lower envelopes of the γ wave at each time. The feature amount extractor 103 extracts an α wave band signal that is the signal in the frequency band of the α wave from an envelope signal using a band-pass filter through which the frequency band of the α wave is passed. The α wave band signal is a signal indicating the change of the amplitude of the γ wave in the frequency band of the α wave.

The feature amount extractor 103 applies Hilbert transformation to the signal of the α wave to acquire the phase of the signal of the α wave. Moreover, the feature amount extractor 103 applies Hilbert transformation to an α wave band signal to acquire the phase of the α wave band signal. The feature amount extractor 103 calculates SI that is a concordance rate on the basis of the following equation (2) in a case in which the phase of the signal of the α wave at time n is φ_{ω} (n), and the phase of the α wave band signal at time n is φ_{γω} (n). The concordance rate corresponds to the value of the coupling of the phase of the α wave and the amplitude of the γ wave. The SI is a value of 0 or more and 1 or less. The larger SI means that the amplitude of the γ wave is in conjunction with the phase of the α wave.

$$[ Math 2 ]$$

$$SI = \vert \frac{1}{N} {\sum }_{t = 1}^{N} exp ( i ( {ϕ}_{ω} ( n ) - {ϕ}_{γ ω} ( n ) ) ) \vert \qquad ( 2 )$$

The value of the coupling of the phase of the θ wave and the amplitude of the γ wave of the brain wave at a specific measurement spot may also be adopted as the feature amount. In this case, the θ wave is a low-frequency component, and the γ wave is a high-frequency component. The value of the coupling of the phase of the δ wave and the amplitude of the γ wave of the brain wave at a specific measurement spot may be adopted as the feature amount. In this case, the δ wave is a low-frequency component, and the γ wave is a high-frequency component.

The value of the coupling of the phase of the α wave and the amplitude of the β wave of the brain wave at a specific measurement spot may also be adopted as the feature amount. In this case, the α wave is a low-frequency component, and the β wave is a high-frequency component. The value of the coupling of the phase of the θ wave and the amplitude of the β wave of the brain wave at a specific measurement spot may also be adopted as the feature amount. In this case, the θ wave is a low-frequency component, and the β wave is a high-frequency component. The value of the coupling of the phase of the δ wave and the amplitude of the β wave of the brain wave at a specific measurement spot may be adopted as the feature amount. In this case, the δ wave is a low-frequency component, and the β wave is a high-frequency component.

The feature amount extractor 103 calculates the value of the coupling of the phase of the θ wave and the amplitude of the γ wave, the value of the coupling of the phase of the δ wave and the amplitude of the γ wave, the value of the coupling of the phase of the α wave and the amplitude of the β wave, the value of the coupling of the phase of the θ wave and the amplitude of the β wave, and the value of the coupling of the phase of the δ wave and the amplitude of the β wave by a method similar to that in the case of calculating the value of the coupling of the phase of the α wave and the amplitude of the γ wave. With regard to the six kinds of the coupling described above, the values of the coupling and variations in the values of the coupling are considered to differ between a person with a mental disorder and a person with no mental disorder. Thus, the values of the six kinds of the coupling described above are adopted as the feature amounts.

In the present embodiment, the feature amount extractor 103 extracts, as the feature amounts, the values of the frequency powers of the α, β, γ, θ, and δ waves of the brain wave at each measurement spot, the phase synchronization value of the α, β, γ, θ, and δ waves of each brain wave between the measurement spots, the value of the coupling of the phase of the α wave and the amplitude of the γ wave of the brain wave at a specific measurement spot, the value of the coupling of the phase of the θ wave and the amplitude of the γ wave of the brain wave at a specific measurement spot, the value of the coupling of the phase of the δ wave and the amplitude of the γ wave of the brain wave at a specific measurement spot, the value of the coupling of the phase of the α wave and the amplitude of the β wave of the brain wave at a specific measurement spot, the value of the coupling of the phase of the θ wave and the amplitude of the β wave of the brain wave at a specific measurement spot, and the value of the coupling of the phase of the δ wave the amplitude of the β wave of the brain wave at a specific measurement spot. The feature amount extractor 103 is one example of feature amount extractors.

The mental disorder determiner 104 determines whether or not a subject has a predetermined mental disorder on the basis of a feature amount variation. The feature amount variation is a differential value between a feature amount extracted from a pre-stimulus brain wave that is a brain wave measured immediately before a magnetic stimulation and a feature amount extracted from a post-stimulus brain wave that is a brain wave measured immediately after the magnetic stimulation. The feature amount variation is considered to differ between a case in which the subject has a predetermined mental disorder and a case in which the subject has no predetermined mental disorder. Thus, the mental disorder determiner 104 determines whether or not the subject has the predetermined mental disorder on the basis of the feature amount variation.

The feature amount extracted from the pre-stimulus brain wave is also considered to differ between the case in which the subject has the predetermined mental disorder and the case in which the subject has no predetermined mental disorder. The feature amount extracted from the post-stimulus brain wave is also considered to differ between the case in which the subject has the predetermined mental disorder and the case in which the subject has no predetermined mental disorder. Thus, in the present embodiment, the mental disorder determiner 104 determines whether or not the subject has the predetermined mental disorder on the basis of the feature amount extracted from the pre-stimulus brain wave and the feature amount extracted from the post-stimulus brain wave as well as the feature amount variation.

A method in which the mental disorder determiner 104 determines whether or not the subject has the predetermined mental disorder on the basis of the feature amount variation, the feature amount, and the like can be adjusted as appropriate. For example, the mental disorder determiner 104 may determine whether or not the subject has the predetermined mental disorder using a learned model 121. The learned model 121 is a model into which the feature amount variation, the feature amount extracted from the pre-stimulus brain wave, and the feature amount extracted from the post-stimulus brain wave are input to output the result of determining whether or not the subject has the predetermined mental disorder.

The learned model 121 is a model in which learning is performed using, for example, linear discriminant analysis (LDA), logistic regression, a support vector machine (SVM), or the like. The LDA is a linear discrimination classification method by which a data group is classified so that a within-class variance is minimized and a between-class variance is maximized. The logistic regression is one of multivariate analysis techniques, and one of techniques used in supervised learning. The SVM is one of pattern recognition models using supervised learning. The SVM is an algorithm that executes classification, regression, and the like by determining a boundary line, a hyperplane, or the like by which the two-class data group is divided. The learned model 121 may also be a model in which learning is performed using, for example, k-nearest neighbor algorithm (KNN), naive Bayes (NB), decision tree (DT), random forest (RF), extra tree (ET), light GBM (LG), and the like, as well as the above.

The learned model 121 is generated by, for example, supervised learning. In this case, the learned model 121 includes, as examples, a feature amount extracted from a brain wave and a feature amount variation calculated from the feature amount, and is generated based on many items of training data including, as an answer, information indicating whether or not a person from whom the brain wave is acquired has a predetermined mental disorder. The many items of the training data used in machine learning include a large number of items of training data based on the brain waves of persons with predetermined mental disorders and a large number of items of training data based on the brain waves of persons with no mental disorders. In the present embodiment, the training data includes a feature amount variation, a feature amount extracted from a pre-stimulus brain wave, a feature amount extracted from a post-stimulus brain wave, and information indicating whether or not a person from whom a brain wave is acquired has a mental disorder. The generated learned model 121 is stored in the storage 12.

The mental disorder determiner 104 inputs the feature amount variation, the feature amount extracted from the pre-stimulus brain wave, and the feature amount extracted from the post-stimulus brain wave into the learned model 121, and acquires a determination result output from the learned model 121. The determination result is, for example, a determination result indicating a probability that a subject has a predetermined mental disorder.

The mental disorder determiner 104 determines whether or not a subject has a mental disorder on the basis of the determination result output from the learned model 121. For example, when the probability indicated by the determination result output from the learned model 121 is 50% or more, the mental disorder determiner 104 determines that the subject has the predetermined mental disorder. In contrast, when the probability is less than 50%, the mental disorder determiner 104 determines that the subject does not have the predetermined mental disorder. Alternatively, the mental disorder determiner 104 may adopt, as a determination result, a probability itself indicated by the determination result output from the learned model 121. The mental disorder determiner 104 is one example of mental disorder determiners.

The display controller 105 allows the display 13 to display the determination result whether or not a subject has a predetermined mental disorder. For example, the display controller 105 allows the display 13 to display a presentation screen for presenting the determination result. In the presentation screen, whether or not the subject has the predetermined mental disorder may be presented in categorization of YES/NO, or may be presented as a probability that the subject has the predetermined mental disorder.

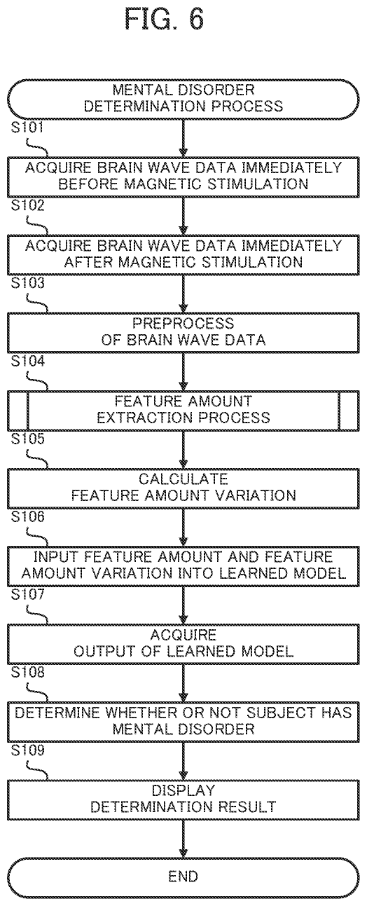

Referring now to FIG. 6, a mental disorder determination process executed by the mental disorder determination device 100 is described. A mental disorder determination support method in the present disclosure is achieved by execution of the mental disorder determination process by the mental disorder determination device 100.

First, the controller 11 included in the mental disorder determination device 100 acquires brain wave data immediately before a magnetic stimulation (step S101). When the process of step S101 is completed, the controller 11 acquires brain wave data immediately after the magnetic stimulation (step S102).

For example, the controller 11 operates the electroencephalograph 300, and then operates the magnetic stimulation device 200 to allow the magnetic stimulation device 200 to execute a periodic magnetic stimulation. The controller 11 acquires brain wave data before and after magnetic stimulations corresponding to all the channels from the electroencephalograph 300 through the first communicator 15. The controller 11 extracts brain wave data immediately before a magnetic stimulation and brain wave data immediately after the magnetic stimulation from the acquired brain wave data on the basis of timing at which the magnetic stimulation device 200 executes the magnetic stimulation.

When the process of step S102 is completed, the controller 11 executes a preprocess for a brain wave data (step S103). The brain wave data acquired by the electroencephalograph 300 may include a noise caused by a magnetic stimulation by the magnetic stimulation device 200, a noise caused by an alternating-current power source, and the like. Thus, the controller 11 removes these noises from the brain wave data, for example, in the preprocess.

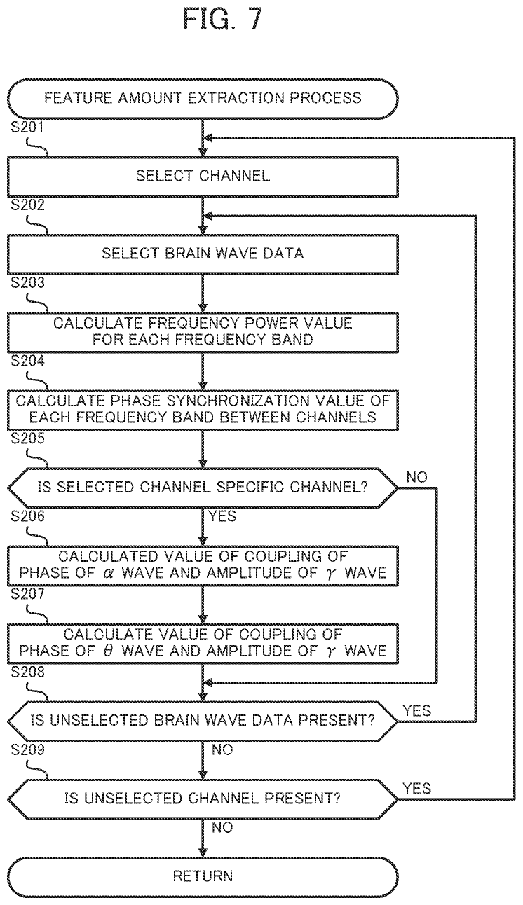

When the process of step S103 is completed, the controller 11 executes a feature amount extraction process (step S104). The feature amount extraction process is described in detail with reference to FIG. 7.

First, the controller 11 selects a channel (step S201). For example, the controller 11 selects one channel from 62 channels. When the process of step S201 is completed, the controller 11 selects brain wave data (step S202). For example, the controller 11 selects one item of brain wave data from brain wave data before a magnetic stimulation and brain wave data after the magnetic stimulation with regard to the selected channel.

When the process of step S202 is completed, the controller 11 calculates a frequency power value for each frequency band with regard to the selected brain wave data (step S203). For example, the controller 11 performs FFT analysis of a time-series voltage signal indicated by the selected brain wave data to determine a power spectrum. The controller 11 calculates the frequency power value for each of α, β, γ, θ, and δ waves from the power spectrum.

When the process of step S203 is completed, the controller 11 calculates the phase synchronization value of each frequency band between channels (step S204). For example, the controller 11 calculates the phase synchronization value of each of the α, β, γ, θ, and δ waves for pairs of all the channels including the selected channel. For example, the controller 11 calculates such values of which the number is 61×5=305. A value that has been already calculated in a process for another channel need not be calculated again.

When the process of step S204 is completed, the controller 11 determines whether or not the selected channel is a specific channel (step S205). The specific channel is a channel corresponding to a site subjected to a magnetic stimulation by the coil 210, and a channel corresponding to the prefrontal area (for example, left dorsolateral prefrontal area).

When determining that the selected channel is the specific channel (step S205: YES), the controller 11 calculates the value of the coupling of the phase of the α wave and the amplitude of the γ wave (step S206). For example, the controller 11 calculates an SI value using Equation (1) as described above, and sets the calculated SI value at the value of the coupling. When the process of step S206 is completed, the controller 11 calculates the value of the coupling of the phase of the θ wave and the amplitude of the γ wave (step S207). For example, the controller 11 calculates the value of the coupling of the phase of the θ wave and the amplitude of the γ wave by a method similar to that in step S206.

When determining that the selected channel is not a specific channel (step S205: NO), or when the process of step S207 is completed, the controller 11 determines whether or not there is unselected brain wave data (step S208). When determining that there is unselected brain wave data (step S208: YES), the controller 11 returns the process to step S202. When determining that there is not unselected brain wave data (step S208: NO), the controller 11 determines whether or not there is an unselected channel (step S209). When determining that there is an unselected channel (step S209: YES), the controller 11 returns the process to step S201. When determining that there is not an unselected channel (step S209: NO), the controller 11 completes the feature amount extraction process.

When the feature amount extraction process of step S104 is completed, the controller 11 calculates a feature amount variation (step S105). For example, the controller 11 calculates a differential value between a feature amount extracted from brain wave data before a magnetic stimulation and a feature amount extracted from brain wave data after the magnetic stimulation on the basis of each channel and each feature amount, and sets the calculated differential value at the feature amount variation.

When the feature amount extraction process of step S105 is completed, the controller 11 inputs the feature amounts and the feature amount variation into the learned model (step S106). In other words, the controller 11 inputs all the extracted feature amounts and all the calculated feature amount variations in the into the learned model. When the process of step S106 is completed, the controller 11 acquires the output of the learned model (step S107).

When the process of step S107 is completed, the controller 11 determines whether or not a subject has a predetermined mental disorder (step S108). For example, the controller 11 determines whether or not the subject has major depressive disorder on the basis of a determination result output from the learned model. When the process of step S108 is completed, the controller 11 displays the determination result (step S109). For example, the controller 11 allows the display 13 to display a presentation screen for presenting the determination result. When the process of step S109 is completed, the controller 11 completes the mental disorder determination process.

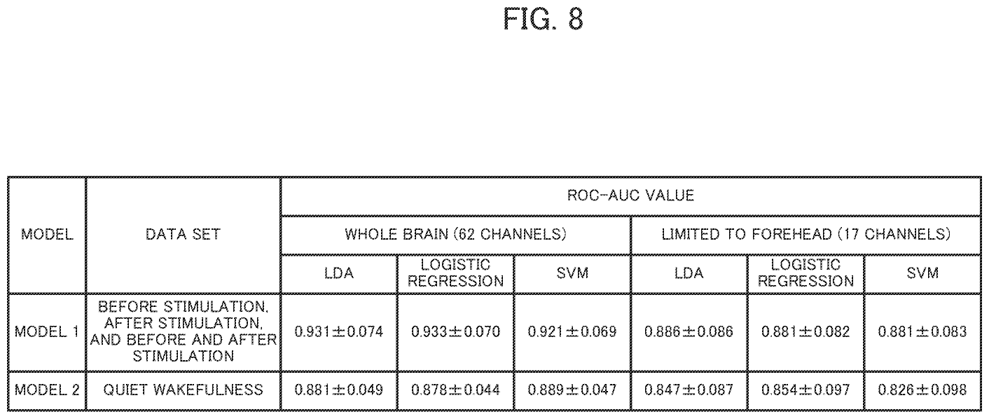

Subsequently, the results of evaluation of the performance of the learned model according to the present embodiment are described. FIG. 8 illustrates the results of evaluation of the performance of actually generated models 1 and 2 using receiver operating characteristic (ROC)-area under curve (AUC) values. The model 1 is a learned model according to the present embodiment, and the model 2 is a learned model according to Comparative Example. Such an ROC-AUC value is a value used in evaluation of the performance of a model, and such a value closer to 1 means such a model with higher performance.

In the present evaluation, (1) the frequency power values (for all the channels) of α, β, γ, θ, and δ waves, (2) the phase synchronization values (for all the channels) of the α, β, γ, θ, and δ waves, (3) the coupling value (only for a specific channel) of the phase of the α wave and the amplitude of the γ wave, and (4) the coupling value (only for a specific channel) of the phase of the θ wave and the amplitude of the γ wave were adopted as feature amounts. In the present evaluation, 62 channels (channels for the whole brain) and 17 channels (channels for the frontal lobe) were adopted with regard to the number of the channels. In the present evaluation, LDA, logistic regression, and SVM were adopted as learning methods in a machine learning model.

In such a case, the model 1 is a learned model in which the feature amount of a pre-stimulus brain wave, the feature amount of a post-stimulus brain wave, and a feature amount variation that is a differential value between the feature amount of the pre-stimulus brain wave and the feature amount of the post-stimulus brain wave, are adopted as data set. Such a feature amount is extracted from the mean waveform of brain waves before and after the first to 80th magnetic stimulations. The model 2 is a learned model in which the feature amount of a quiet wakefulness brain wave is adopted as a data set.

In the present evaluation, the performance of the models 1 and 2 were evaluated for, as a subject, each of 60 patients with depression, diagnosed as treatment-resistant depression by a psychiatric specialist, and 60 healthy individuals with no mental disorder. The ROC-AUC values that are the evaluation results are as illustrated in FIG. 8.

In other words, in the model 1, LDA is 0.931, logistic regression is 0.933, and SVM is 0.921 in the case of the 62 channels, and LDA is 0.886, logistic regression is 0.881, and SVM is 0.881 in the case of the 17 channels. In contrast, in the model 2, LDA is 0.881, logistic regression is 0.878, and SVM is 0.889 in the case of the 62 channels, and LDA is 0.847, logistic regression is 0.854, and SVM is 0.826 in the case of the 17 channels.

In consideration of the evaluation results, the more favorable ROC-AUC values were recorded in the model 1 using the feature amount variation than in the model 2 using only the feature amount in quiet wakefulness, and therefore, the model 1 is considered to be more suitable for determining depression than the model 2. There are no large differences in the ROC-AUC values between a case in which the number of channels is 62 and a case in which the number of channels is 17. Therefore, depression is considered to be able to be accurately determined even from the brain waves of the 17 channels in the forehead. Since stable ROC-AUC values are obtained regardless of the learning method of the machine learning model, the general-purpose properties of the model can be expected.

As described above, in the present embodiment, whether or not a subject has a predetermined mental disorder is determined on the basis of a feature amount variation that is a differential value between a feature amount extracted from a brain wave measured immediately before a magnetic stimulation and a feature amount extracted from a brain wave measured immediately after the magnetic stimulation. In such a case, a feature amount variation is considered to differ between a person with a predetermined mental disorder and a person with no mental disorder. Accordingly, the presence or absence of a predetermined mental disorder can be accurately determined in accordance with the present embodiment.

In the present embodiment, the feature amount variation is a differential value between a feature amount extracted from a brain wave measured immediately before the second or later magnetic stimulation and a feature amount extracted from a brain wave measured immediately after the second or later magnetic stimulation. The brain waves measured before and after the second or later magnetic stimulation is considered to be more stable than the brain waves measured before and after the first magnetic stimulation. Accordingly, more accurate determination of the presence or absence of a predetermined mental disorder (for example, major depressive disorder) can be expected in accordance with the present embodiment.

In the present embodiment, a frequency power value at each measurement spot and in each frequency band, a phase synchronization value in each frequency band between the measurement spots, the value of the coupling of the phase of the α wave and the amplitude of the γ wave of a brain wave at a specific measurement spot, the value of the coupling of the phase of the θ wave and the amplitude of the γ wave of the brain wave at the specific measurement spot, the value of the coupling of the phase of the δ wave and the amplitude of the γ wave of the brain wave at the specific measurement spot, the value of the coupling of the phase of the α wave and the amplitude of the β wave of the brain wave at the specific measurement spot, the value of the coupling of the phase of the θ wave and the amplitude of the β wave of the brain wave at the specific measurement spot, and the value of the coupling of the phase of the δ wave the amplitude of the β wave of the brain wave at the specific measurement spot are adopted as feature amounts. The variations in the feature amounts are considered to differ between a person with major depressive disorder and a person with no major depressive disorder. Accordingly, more accurate determination of the presence or absence of major depressive disorder can be expected in accordance with the present embodiment.

In the present embodiment, a case in which the mental disorder is a major depressive disorder or treatment-resistant depression is imagined. Feature amount variations are considered to differ between persons who have and do not have these mental disorders. Accordingly, the presence or absence of the predetermined mental disorder can be accurately determined in accordance with the present embodiment.

In the present embodiment, whether or not a subject has a mental disorder is determined using a learned model into which a feature amount variation is input to output the result of determining whether or not a subject has a predetermined mental disorder. Machine learning by enormous volumes of training data is considered to result in the high determination accuracy of the learned model. Accordingly, the presence or absence of the predetermined mental disorder can be more accurately determined in accordance with the present embodiment.

In the present embodiment, whether or not a subject has a mental disorder is determined on the basis of a feature amount variation, a feature amount extracted from a brain wave measured immediately before a magnetic stimulation, and a feature amount extracted from a brain wave measured immediately after the magnetic stimulation. Not only the feature amount variation but also the feature amounts themselves are considered to differ between a person with a mental disorder and a person with no mental disorder. Accordingly, a more accurate determination of the presence or absence of the predetermined mental disorder can be expected in accordance with the present embodiment.

### Embodiment 2

In Embodiment 1, the example in which the mental disorder determination device 100 executes the processes such as the control of the magnetic stimulation device 200 and the electroencephalograph 300 and the display of the determination result is described. In the present embodiment, an example in which other devices execute the processes is described. Description of configurations and functions similar to those in Embodiment 1 is omitted or simplified as appropriate.

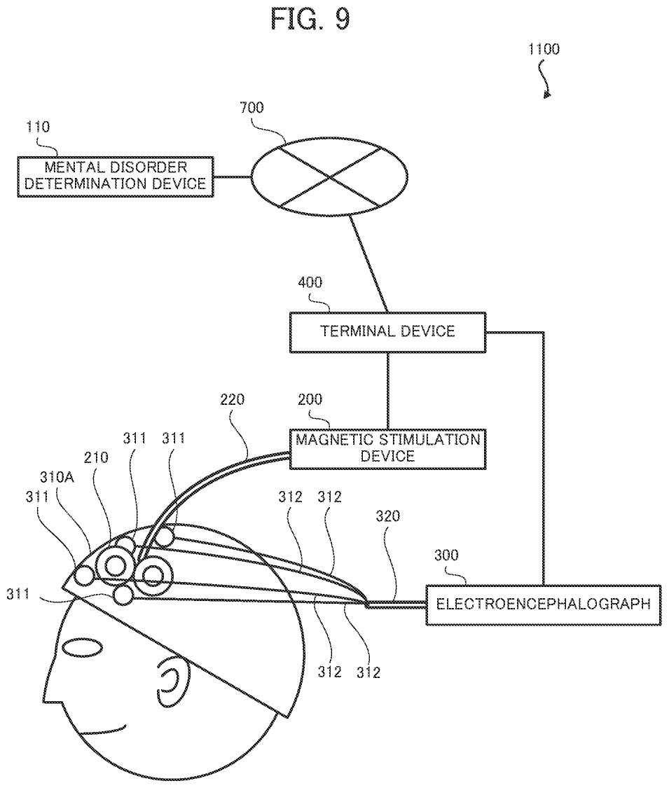

As illustrated in FIG. 9, the mental disorder determination system 1100 according to the present embodiment includes a mental disorder determination device 110, a magnetic stimulation device 200, an electroencephalograph 300, and a terminal device 400. The mental disorder determination device 110 and the terminal device 400 are connected to each other through a communication network 700. The communication network 700 is, for example, the Internet.

The mental disorder determination device 110 is a device that determines whether or not a subject has a predetermined mental disorder on the basis of the brain wave of the subject. Unlike the mental disorder determination device 100, however, the mental disorder determination device 110 does not control the magnetic stimulation device 200 and the electroencephalograph 300, and does not display a determination result. Instead, the mental disorder determination device 110 acquires brain wave data from the terminal device 400 through the communication network 700, and transmits the determination result to the terminal device 400 through the communication network 700.

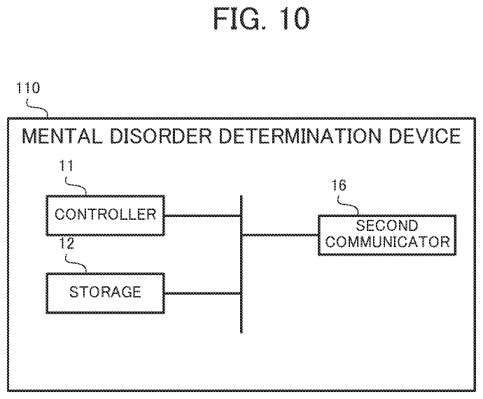

The mental disorder determination device 110 is, for example, a cloud server that provides mental disorder determination service. The cloud server is a server that provides resources in cloud computing. The mental disorder determination device 110 is one example of servers. As illustrated in FIG. 10, the mental disorder determination device 110 includes a controller 11, a storage 12, and a second communicator 16.

The second communicator 16 communicates with various devices connected to the communication network 700 under control by the controller 11. In the present embodiment, the second communicator 16 communicates with the terminal device 400 under control by the controller 11. The second communicator 16 includes a communication interface for connection to the communication network 700.

The magnetic stimulation device 200 is a device that applies transcranial magnetic stimulation to a subject. In the present embodiment, the magnetic stimulation device 200 is controlled by the terminal device 400. The electroencephalograph 300 is a device that measures the brain wave of a subject. In the present embodiment, the electroencephalograph 300 is controlled by the terminal device 400. In the present embodiment, the electroencephalograph 300 is connected to an electroencephalography cap 310A through a cable 320.

The electroencephalography cap 310A is a cap that is attached to the head of a subject in order to measure the brain wave of the subject. The number of placed electrodes 311 and spots at which the electrodes 311 are placed on the electroencephalography cap 310A are different from those on the electroencephalography cap 310. In other words, the plurality of electrodes 311 are placed on the portion, corresponding to the forehead (for example, frontal lobe), of the electroencephalography cap 310A, and the brain wave corresponding to the forehead (for example, frontal lobe) of the subject is measured. In the present embodiment, the number of the electrodes 311, measurement spots, and channels is 17.

The terminal device 400 is a device that executes various processes associated with a mental disorder determination process. For example, the terminal device 400 controls the magnetic stimulation device 200 to apply a magnetic stimulation to the brain of a subject. The terminal device 400 acquires brain wave data indicating the brain wave of the subject from the electroencephalograph 300. The terminal device 400 transmits the brain wave data, acquired from the electroencephalograph 300, to the mental disorder determination device 110 through the communication network 700. The terminal device 400 presents a determination result, acquired from the mental disorder determination device 110, through the communication network 700.

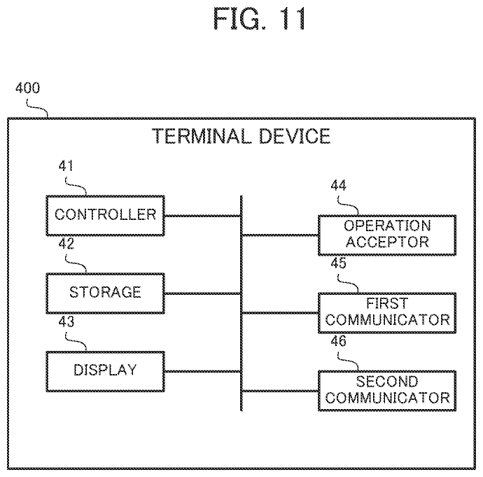

The terminal device 400 is, for example, a personal computer, a smartphone, a tablet terminal, or the like. The terminal device 400 is one example of terminal devices. As illustrated in FIG. 11, the terminal device 400 includes a controller 41, a storage 42, a display 43, an operation acceptor 44, a first communicator 45, and a second communicator 46.

The controller 41 includes a CPU, a ROM, a RAM, an RTC, and the like. The CPU is also referred to as a central processing unit, a central arithmetic device, a processor, a microprocessor, a microcomputer, a DSP, or the like, and functions as a central arithmetic processing device that executes a process and arithmetic according to control of the terminal device 400. In the controller 41, the CPU reads a program and data stored in the ROM, and integrally controls the terminal device 400 using the RAM as a work area. For example, the RTC is an integrated circuit including a time measurement function. The CPU can specify current date and time on the basis of time information read from the RTC.

The storage 42 includes a nonvolatile semiconductor memory such as a flash memory, an EPROM, or an EEPROM, and plays a role as a so-called auxiliary storage device. The storage 42 stores a program and data used for allowing the controller 41 to execute various processes. Moreover, the storage 42 stores data generated and acquired by the execution of the various processes by the controller 41.

The display 43 displays various images under control by the controller 41. For example, the display 43 displays a screen for accepting various operations from a user. The display 43 includes a touch screen, a liquid crystal display, and/or the like. The operation acceptor 44 accepts the various operations from the user, and supplies information indicating the contents of the accepted operations to the controller 41. The operation acceptor 44 includes a touch screen, a button, and a lever, and/or the like.

The first communicator 45 communicates with various devices under control by the controller 41. In the present embodiment, the first communicator 45 communicates with the magnetic stimulation device 200 and the electroencephalograph 300 under control by the controller 41. The first communicator 45 communicates with various devices in conformity with a well-known wire communication standard or a well-known wireless communication standard. The first communicator 45 includes a communication interface conforming to various communication standards.

The second communicator 46 communicates with various device connected to the communication network 700 under control by the controller 41. In the present embodiment, the second communicator 46 communicates with the mental disorder determination device 110 under control by the controller 41. The second communicator 46 includes a communication interface for connection to the communication network 700.

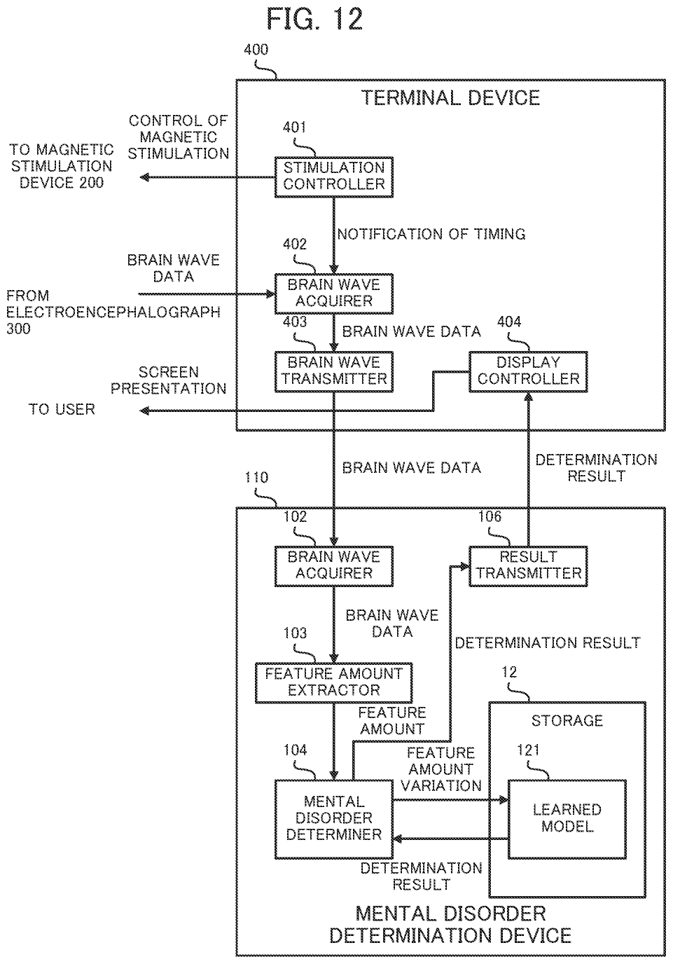

Referring now to FIG. 12, the functions of the mental disorder determination device 110 and the terminal device 400 are described. The mental disorder determination device 110 functionally includes a brain wave acquirer 102, a feature amount extractor 103, a mental disorder determiner 104, and a result transmitter 106. The terminal device 400 functionally includes a stimulation controller 401, a brain wave acquirer 402, a brain wave transmitter 403, and a display controller 404. Each function thereof is implemented by software, firmware, or a combination of software and firmware. The software and the firmware are described as programs, and stored in ROM, the storage 12, or the storage 42. The CPU executes the programs stored in the ROM, the storage 12, or the storage 42, thereby implementing each function thereof.

The stimulation controller 401 controls a magnetic stimulation to the brain of a subject. For example, the stimulation controller 401 controls the magnetic stimulation device 200 to apply magnetic stimulations to the brain of the subject at predetermined time intervals. The operation of the stimulation controller 401 is basically similar to the operation of the stimulation controller 101.

The brain wave acquirer 402 acquires the brain wave of the subject. For example, the brain wave acquirer 402 controls the electroencephalograph 300 to measure the brain wave of the subject, and acquires brain wave data indicating the measured brain wave from the electroencephalograph 300. The operation of the brain wave acquirer 402 is basically similar to the operation of the brain wave acquirer 102. The brain wave acquirer 402 is one example of brain wave acquirers.

The brain wave transmitter 403 transmits brain wave data acquired by the brain wave acquirer 402 to the mental disorder determination device 110 through the communication network 700. The brain wave transmitter 403 is one example of brain wave transmitters. The brain wave acquirer 102 acquires the brain wave of the subject. For example, the brain wave acquirer 102 acquires brain wave data indicating the brain wave of the subject from the terminal device 400 through the communication network 700.

The feature amount extractor 103 extracts a feature amount representing the feature of the brain wave from the brain wave of the subject, the brain wave being measured when a magnetic stimulation is applied to the brain of the subject. In other words, the feature amount extractor 103 extracts a feature amount representing the feature of the brain wave of the subject from the brain wave indicated by the brain wave data acquired by the brain wave acquirer 102.

The mental disorder determiner 104 determines whether or not the subject has a mental disorder on the basis of a feature amount variation. For example, the mental disorder determiner 104 determines whether or not the subject has a mental disorder using a learned model 121. The result transmitter 106 transmits a determination result provided by the mental disorder determiner 104 to the terminal device 400 through the communication network 700. The result transmitter 106 is one example of transmitters.

A display controller 105 allows a display 13 to display the result of determining whether or not the subject has a mental disorder. For example, the display controller 105 acquires a determination result from the mental disorder determination device 110 through the communication network 700, and allows the display 43 to display a presentation screen for presenting the determination result. The display controller 105 is one example of display controllers. The display 43 is one example of displays.

In the present embodiment, instead of the mental disorder determination device 110, the terminal device 400 executes a process of controlling the magnetic stimulation device 200 and the electroencephalograph 300, a process of displaying a determination result, and the like. The mental disorder determination device 110 that functions as a server that provides mental disorder determination service includes: the feature amount extractor 103 that extracts a feature amount representing the feature of a brain wave from the brain wave of a subject, measured when a magnetic stimulation is applied to the brain of the subject; and the mental disorder determiner 104 that determines whether or not the subject has a predetermined mental disorder on the basis of a feature amount variation that is a differential value between a feature amount extracted from a brain wave measured immediately before the magnetic stimulation and a feature amount extracted from a brain wave measured immediately after the magnetic stimulation. Accordingly, the presence or absence of the predetermined mental disorder can be accurately determined while reducing the processing load of the mental disorder determination device 110 in accordance with the present embodiment.

The brain wave measured in the present embodiment is a brain wave at a plurality of measurement spots on the forehead of a subject, the brain wave being measured through the plurality of electrodes 311 placed on the forehead of a subject. A difference in a brain wave generated from the forehead between a person with a mental disorder and a person with no mental disorder is considered to be more easily confirmed than in brain waves generated from the other sites of the brain. Therefore, even a case in which a brain wave that is used in calculation of a feature amount variation is limited to a brain wave generated from the forehead is considered to be inhibited from resulting in the deterioration of determination accuracy. The case in which the brain wave that is used in the calculation of the feature amount variation is limited to the brain wave generated from the forehead results in suppression of a feature amount to be extracted, a feature amount variation to be calculated, and the like to reduce a processing load for executing a mental disorder determination process. Accordingly, the presence or absence of a mental disorder can be accurately determined while reducing the processing load of the mental disorder determination device 110 in accordance with the present embodiment.

### Alternative Example

The embodiments of the present disclosure have been described above. However, modifications and applications according to various forms can be made.

It is optional to adopt which one of the structures, functions, and operations described in the embodiments described above. In addition to the structures, functions, and operations described above, further structures, functions, and operations may also be adopted. The embodiments described above can be freely combined as appropriate. The numbers of the components described in the embodiments described above can be adjusted as appropriate. It will be appreciated that materials, sizes, electrical characteristics, and the like that can be adopted are not limited to those described in the embodiments described above.

In Embodiment 1, an example is described in which feature amounts include the frequency power value of the α, β, γ, θ, and δ waves of a brain wave at each measurement spot, the phase synchronization value of the α, β, γ, θ, and δ waves of each brain wave between the measurement spots, the value of the coupling of the phase of the α wave and the amplitude of the γ wave of the brain wave at a specific measurement spot, and the value of the coupling of the phase of the θ wave and the amplitude of the γ wave of the brain wave at a specific measurement spot. Such a feature amount may include at least one of the frequency power value described above, the phase synchronization value described above, and the two coupling values described above.

The above-described frequency power values and phase synchronization values of all of the α, β, γ, θ, and δ waves need not be determined as the feature amounts. For example, the above-described frequency power value and phase synchronization value of at least one of the α, β, γ, θ, and δ waves may be determined as the feature amounts. The more kinds of adopted feature amounts can be expected to result in improvement in accuracy.

In Embodiment 1, an example is described in which whether or not a subject has a predetermined mental disorder is determined based on a feature amount variation, a feature amount extracted from a pre-stimulus brain wave, and a feature amount extracted from a post-stimulus brain wave. A feature amount extracted from a quiet wakefulness brain wave may be further used for the determination, and the feature amount extracted from the pre-stimulus brain wave or the feature amount extracted from the post-stimulus brain wave need not be used for the determination.

For example, only a feature amount variation may be used for the determination. Alternatively, the feature amount variation and a feature amount extracted from a quiet wakefulness brain wave may be used for determination. Alternatively, the feature amount variation and a feature amount extracted from a pre-stimulus brain wave may be used for the determination. Alternatively, the feature amount variation and a feature amount extracted from a post-stimulus brain wave may be used for the determination.

Alternatively, a feature amount variation, a feature amount extracted from a quiet wakefulness brain wave, and a feature amount extracted from a pre-stimulus brain wave may be used for the determination. Alternatively, the feature amount variation, the feature amount extracted from a quiet wakefulness brain wave, and a feature amount extracted from a post-stimulus brain wave may be used for the determination. Alternatively, the feature amount variation, the feature amount extracted from the quiet wakefulness brain wave, a feature amount extracted from a pre-stimulus brain wave, and the feature amount extracted from the post-stimulus brain wave may be used for the determination. In the case of using a learned model, a learned model into which a feature amount that is used for the determination as well as a feature amount variation that is used for the determination is input to output the result of determining whether or not a subject has a predetermined mental disorder is adopted.

In Embodiment 1, an example is described in which a magnetic stimulation is repeatedly applied to the brain of a subject, and brain waves before and after the application of the second or later magnetic stimulation are used for determining a predetermined mental disorder. A brain wave used for determining the presence or absence of the predetermined mental disorder is not limited to the example. For example, a magnetic stimulation may be repeatedly applied to the brain of the subject, and a brain wave before and after the application of the first magnetic stimulation may be used for determining the mental disorder. A single magnetic stimulation may be applied to the brain of the subject, and brain waves before and after the application of the magnetic stimulation may be used for determining the presence or absence of the predetermined mental disorder.

In Embodiment 1, an example is described in which the pre-stimulus brain wave is a brain wave between 1550 msec before a magnetic stimulation and 50 msec before the magnetic stimulation, and the post-stimulus brain wave is a brain wave between 50 msec after a magnetic stimulation and 550 msec after the magnetic stimulation. The pre-stimulus brain wave, the post-stimulus brain wave, and the like are not limited to the example. For example, the pre-stimulus brain wave may be a brain wave from 2000 msec before a magnetic stimulation to the time of the magnetic stimulation, and the post-stimulus brain wave may be a brain wave between 15 msec after a magnetic stimulation and 2000 msec after the magnetic stimulation.

In the Embodiment 1, an example is described in which the mental disorder determination device 100, the magnetic stimulation device 200, and the electroencephalograph 300 are separate devices. A device into which these devices are integrated may also be adopted. For example, the mental disorder determination device 100 may be allowed to include the function of the magnetic stimulation device 200, the mental disorder determination device 100 may be allowed to include the function of the electroencephalograph 300, or the mental disorder determination device 100 may be allowed to include the functions of the magnetic stimulation device 200 and the electroencephalograph 300.

In the embodiments described above, the controller 11 or 41 functions as each section illustrated in FIG. 5 or 11 by execution of the program stored in the ROM or the storage 12 or 42 by the CPU. In the present disclosure, however, the controller 11 or 41 may be dedicated hardware. The dedicated hardware is, for example, a single circuit, a composited circuit, a programed processor, an application specific integrated circuit (ASIC), a field-programmable gate array (FPGA), a combination thereof, or the like. In a case in which the controller 11 or 41 is dedicated hardware, each function of each section may be implemented by separate hardware, or the functions of each section may be collectively implemented by single hardware. Some of the functions of each section may be achieved by dedicated hardware, and the other may be achieved by software or firmware. In such a manner, the controller 11 or 41 can implement each of the functions described above by hardware, software, firmware, or a combination thereof.

Application of operation programs that define the operations of the mental disorder determination devices 100 and 110 and the terminal device 400 according to the present disclosure to a computer such as an existing personal computer or an information terminal device also enables the computer to function as the mental disorder determination device 100 or 110 or the terminal device 400 according to the present disclosure. A method of distributing such a program is optional, and the program stored in a non-transitory computer-readable recording medium such as, for example, a compact disk ROM (CD-ROM), a digital versatile disk (DVD), a magneto optical disk (MO), or a memory card may be distributed, or the program may be distributed through a communication network such as the Internet.

As described above, the technology according to the present disclosure is protected in various forms. Examples of conceivable protected forms include servers, non-transitory recording media, program products, classifiers, and neural networks as well as mental disorder determination devices, learned models, mental disorder determination assistance methods, programs, terminal devices. Such a non-transitory recording medium is, for example, a non-transitory recording medium that stores a program that allows a computer to function as the feature amount extractor 103 and the mental disorder determiner 104. The non-transitory recording medium described above may be a non-transitory recording medium that stores the program described above. Such a program product is a product related to the program described above. The program product includes a product without any non-transitory recording medium. The above-described program and the above-described program product may be forms provided by non-transitory recording media, or may be forms delivered by an external computer.

The above-described classifier is a classifier corresponding to the learned model 121. In other words, the above-described classifier is a classifier into which a feature amount variation that is a differential value between a feature amount extracted from a brain wave measured immediately before a magnetic stimulation to the brain of a subject and a feature amount extracted from a brain wave measured immediately after the magnetic stimulation is input to output the result of determining whether or not the subject has a predetermined mental disorder.

The above-described neural network is a neural network corresponding to the learned model 121. In other words, the above-described neural network is a neural network into which a feature amount variation that is a differential value between a feature amount extracted from a brain wave measured immediately before a magnetic stimulation to the brain of a subject and a feature amount extracted from a brain wave measured immediately after the magnetic stimulation is input to output the result of determining whether or not the subject has a predetermined mental disorder.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims. along with the full range of equivalents to which such claims are entitled.

1. A mental disorder determination device, comprising:
processing circuitry to
extract a feature amount that represents a feature of a brain wave of a subject from the brain wave measured when a magnetic stimulation is applied to a brain of the subject, and
determine whether or not the subject has a predetermined mental disorder based on a feature amount variation that is a differential value between a feature amount extracted from a brain wave measured immediately before the magnetic stimulation and a feature amount extracted from a brain wave measured immediately after the magnetic stimulation.

2. The mental disorder determination device according to claim 1, wherein
the magnetic stimulation is consecutively applied to the brain of the subject, and
the feature amount variation is a differential value between a feature amount extracted from a brain wave measured immediately before a second or later magnetic stimulation and a feature amount extracted from a brain wave measured immediately after the second or later magnetic stimulation.

3. The mental disorder determination device according to claim 1, wherein
the brain waves are brain waves at a plurality of measurement spots of the subject, and
the feature amount includes at least one of a frequency power value of at least one of α, β, γ, θ, and δ waves of each brain wave at the measurement spots, a phase synchronization value of at least one of α, β, γ, θ, and δ waves of each brain wave between the measurement spots, or a phase-amplitude coupling value in at least one combination of a plurality of combinations of a phase of an α, θ, or δ wave and an amplitude of a β or γ wave of a brain wave at a specific measurement spot.

4. The mental disorder determination device according to claim 1, wherein
the brain waves are brain waves at a plurality of measurement spots of the subject, and
the feature amount includes a frequency power value of at least one of α, β, γ, θ, and δ waves of each brain wave at the measurement spots, a phase synchronization value of at least one of α, β, γ, θ, and δ waves of each brain wave between the measurement spots, and a phase-amplitude coupling value in at least one combination of a plurality of combinations of a phase of an α, θ, or δ wave and an amplitude of a β or γ wave of a brain wave at a specific measurement spot.

5. The mental disorder determination device according to claim 1, wherein the mental disorder is major depressive disorder or treatment-resistant depression.

6. The mental disorder determination device according to claim 1, wherein the brain waves are brain waves at a plurality of measurement spots on a forehead of the subject, the brain waves being measured through a plurality of electrodes placed on the forehead of the subject.

7. The mental disorder determination device according to claim 1, wherein the processing circuitry determines whether or not the subject has the mental disorder using a learned model into which the feature amount variation is input to output a result of determining whether or not the subject has the mental disorder.

8. The mental disorder determination device according to claim 1, wherein the processing circuitry determines whether or not the subject has the mental disorder based on the feature amount variation and on a feature amount extracted from a brain wave measured in quiet wakefulness.

9. The mental disorder determination device according to claim 1, wherein the processing circuitry determines whether or not the subject has the mental disorder based on the feature amount variation and on a feature amount extracted from a brain wave measured immediately before the magnetic stimulation.

10. The mental disorder determination device according to claim 1, wherein the processing circuitry determines whether or not the subject has the mental disorder based on the feature amount variation and on a feature amount extracted from a brain wave measured immediately after the magnetic stimulation.

11. The mental disorder determination device according to claim 1, wherein the processing circuitry determines whether or not the subject has the mental disorder based on the feature amount variation, on a feature amount extracted from a brain wave measured in quiet wakefulness, and on a feature amount extracted from a brain wave measured immediately before the magnetic stimulation.

12. The mental disorder determination device according to claim 1, wherein the processing circuitry determines whether or not the subject has the mental disorder based on the feature amount variation, on a feature amount extracted from a brain wave measured in quiet wakefulness, and on a feature amount extracted from a brain wave measured immediately after the magnetic stimulation.

13. The mental disorder determination device according to claim 1, wherein the processing circuitry determines whether or not the subject has the mental disorder based on the feature amount variation, on a feature amount extracted from a brain wave measured immediately before the magnetic stimulation, and on a feature amount extracted from a brain wave measured immediately after the magnetic stimulation.

14. The mental disorder determination device according to claim 1, wherein the processing circuitry determines whether or not the subject has the mental disorder based on the feature amount variation, on a feature amount extracted from a brain wave measured in quiet wakefulness, on a feature amount extracted from a brain wave measured immediately before the magnetic stimulation, and on a feature amount extracted from a brain wave measured immediately after the magnetic stimulation.

15. A terminal device, comprising:
processing circuitry to
acquire brain wave data indicating a brain wave of a subject, the brain wave being measured when a magnetic stimulation is applied to the brain of the subject,
transmit the acquired brain wave data to a server, and
cause a display to display a result of determining whether or not the subject has a predetermined mental disorder, the result being determined, by the server, based on a feature amount variation that is a differential value between a feature amount extracted from a brain wave measured immediately before the magnetic stimulation and a feature amount extracted from a brain wave measured immediately after the magnetic stimulation.

16. A classifier, to:
receive a feature amount variation that is a differential value between a feature amount extracted from a brain wave measured immediately before a magnetic stimulation to a brain of a subject and a feature amount extracted from a brain wave measured immediately after the magnetic stimulation; and
output a result of determining whether or not the subject has a predetermined mental disorder.

17. A mental disorder determination assistance method for assisting determination of presence or absence of a mental disorder, the method comprising:
extracting a feature amount representing a feature of a brain wave of a subject from the brain wave measured when a magnetic stimulation is applied to a brain of the subject, and
determining whether or not the subject has a predetermined mental disorder based on a feature amount variation that is a differential value between a feature amount extracted from a brain wave measured immediately before the magnetic stimulation and a feature amount extracted from a brain wave measured immediately after the magnetic stimulation.

18. A non-transitory computer-readable recording medium storing a program, the program causing a computer to function as
a feature amount extractor that extracts a feature amount representing a feature of a brain wave of a subject from the brain wave measured when a magnetic stimulation is applied to a brain of the subject, and
a mental disorder determiner that determines whether or not the subject has a predetermined mental disorder based on a feature amount variation that is a differential value between a feature amount extracted from a brain wave measured immediately before the magnetic stimulation and a feature amount extracted from a brain wave measured immediately after the magnetic stimulation.
